(19) United States
(12) Patent Application Publication
(10) Pub. No.: US 2026/0355113 A1
(43) Pub. Date: Nov. 12, 2026

(54) CATIONIC MICRO-VESICLES AND FUNCTIONALIZED EXOSOME-LIKE VECTORS FOR THE DELIVERY OF FUNCTIONAL AGENT TO THE SKIN

(71) Applicant: Coty Inc., New York, NY (US)

(72) Inventors: Muriel Pujos, New York, NY (US); Olivier Doucet, New York, NY (US); Cecile Chamayou-Robert, New York, NY (US)

(21) Appl. No.: 19/715,450

(22) Filed: Jun. 22, 2026

Related U.S. Application Data
(63) Continuation of application No. PCT/US2024/060548, filed on Dec. 17, 2024.

(30) Foreign Application Priority Data
Dec. 22, 2023 (FR) 2315131

(51) Int. Cl. A61K 9/1271; A61K 31/4045

(52) U.S. Cl. CPC A61K 9/1271; A61K 31/4045

(57) ABSTRACT

The present invention relates to liposomes displaying cationic charges on their outer surface. The liposomes can be functionalized with ligands through electrostatic interactions with the positive charges to give exosome-like vectors. Both the micro-vesicles and the exosome-like vectors are intended to be used to deliver functional (active) agents to cells, in particular those of the skin, for both cosmetic and therapeutic treatments.

### CROSS-REFERENCE TO RELATED APPLICATIONS

This application is a continuation of PCT Application Serial No. PCT/US2024/060548, filed Dec. 17, 2024, which application claims priority to French Application Serial No. 2315131, filed Dec. 22, 2023, which are incorporated by reference herein in their entireties.

### FIELD OF THE INVENTION

The present invention discloses micro-vesicles enveloped by a membranous lipid bilayer and displaying positive charges on their outer surface. The micro-vesicles are also known as positively charged liposomes and the two terms will be used interchangeably herein.

It also discloses exosome-like vectors obtained by functionalizing the surface of the membranous lipid bilayer of the cationic micro-vesicles with ligands such as oligopeptide-1 or oligopeptide-2. Functionalisation occurs through electrostatic interactions between the ligand and the positive charges located on the surface of the micro-vesicles.

Both the micro-vesicles and the exosome-like vectors are intended to be used to deliver functional (active) agents to cells, in particular those of the skin, for both cosmetic and therapeutic treatments.

### BACKGROUND OF THE INVENTION

Skin is the largest organ in the body and covers the body's entire external surface. It is made up of three layers, the epidermis, dermis, and the hypodermis, all three of which vary significantly in their anatomy and function.

Liposomes are micro-vesicles in which an aqueous volume is surrounded by a lipid bilayer membrane. The membrane comprises phospholipids containing a hydrophobic tail and a hydrophilic head, wherein the tails constitute the interior of the membrane and the heads are at the interface between the lipid bilayer and the internal and external aqueous environments respectively. Based on vesicle structure, there are several categories for liposomes: the major types of liposomes are the multilamellar vesicle (MLV, with several lamellar phase lipid bilayers), the small unilamellar liposome vesicle (SUV, with one lipid bilayer) and the large unilamellar vesicle (LUV).

Because they encapsulate an extensive aqueous environment, liposomes can load any kind of hydrophilic functional charge, protect it from the host body metabolic and immunologic attacks and minimize side effects. Additionally, they can also encapsulate lipophilic ingredients in their membrane.

There is, however, an unmet need to develop new micro-vesicle with membranes modified on their outer surface to optimize the targeted delivery of functional agents to cells, in particular skin cells.

Extra-cellular vesicles represent the new frontier in the field of intercellular communication pathways. In particular, exosomes are nano- to micron-sized vesicles budding from cells that display specific organotropic behavior and play a key role in cell-to-cell communication and material transfer pathways. The unique ability of exosomes to interact and be taken up by specific cells makes exosomes an ideal model for the design of ex-novo nano-vesicles as carriers for targeted delivery of functional agents, both in the cosmetic and in the pharmaceutical fields.

Unfortunately, unmodified exosomes, when used for the delivery of active agents, often fail to demonstrate the anticipated results following systemic administration for a number of reasons. Because of this, and since there are numerous similarities between liposomes and exosomes, liposome engineering technologies are being used to engineer exosomes mimetics. The similarities include the fact that both are composed of at least one lipid bilayer, can have a size between 40 and 350 nm and the ability to be loaded with lipophilic and hydrophilic active agents. Furthermore, both can be easily decorated on their surface with targeting ligands.

To date, a number of exosome-like systems have been tested as carriers for active agents with the aim of overcoming the shortcomings of unmodified exosomes. One of such systems is represented by artificial exosome mimetics, which in most cases are liposomes with or without specific proteins in their membranes and are inspired from specific types of exosomes with high organotropism. There is however an unmet need to develop further delivery systems, in particular for the targeted delivery of functional active agents to the skin of a patient or cosmetic care user.

### BRIEF SUMMARY OF THE INVENTION

The present invention is based in part on the observation that micro-vesicles enveloped by a membranous lipid bilayer can be functionalized on their external surface with positive charges by lipids having a positively charged or amphoteric head group. The lipid moiety of the lipid can extend into the membrane bilayer or be absorbed onto the external surface of the membrane bilayer, while the head groups lay on the external surface of the membrane bilayer.

The present invention is further based on the observation that an exosome-like vector can be engineered starting from a positively charged liposome by further functionalizing it with a ligand such as oligopeptide-1 or oligopeptide-2 that are bound to their surface through electrostatic interactions with the positive charges.

### DEFINITIONS

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation.

All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

The term “about” as used herein can allow for a degree of variability in a value or range, for example, within 10%, within 5%, or within 1% of a stated value or of a stated limit of a range and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

When percentages of ingredients in compositions are indicated, the standard error applies. For example, 3.0% is to be interpreted as 2.9% to 3.1%

The term “comprises” and variations thereof is to be interpreted as meaning both “includes”, “substantially consists of” and “consists of”, and variations thereof.

When different embodiments are described that further specify different features of the present invention it is intended that these embodiments can be combined in a further embodiment specifying the two or more corresponding features of the present invention even when such combined embodiment is not explicitly described herein.

When the quantity of an ingredient of a composition is indicated as % it is intended over the total weight of the composition, unless otherwise indicated.

### DETAILED DESCRIPTION OF THE INVENTION

The micro-vesicles according to the invention can be a multilamellar vesicles (MLV), a small unilamellar liposome vesicles (SUV) or a large unilamellar vesicles (LUV). Preferably, they are multilamellar vesicles (MLV).

Multiilamellar vesicles (MLV) can be prepared using conventional techniques, see for example Frank Szoka, Jr. & Demetrios Papahadjopoulos, (1980), “Comparative Properties and Methods of Preparation of Lipid Vesicles (Liposomes)”, Ann. Rev. Biophys. Bioeng., 9:467-508.

The micro-vesicles can be subsequently characterised with the appropriate nanotechnology technique such as DLS/NTA for particle size and M3 PALS (high and low electrophoresis) for zeta potential. When lipophilic ingredients must be added, they are added with lipid molecules at the beginning of the process and dissolved in ethanol.

Average size of micro-vesicles can be determined using Dynamic Light Scattering (DLS) and Nanoparticle Track Analysis (NTA) techniques. Both are based on the Brownian motion of the particle and the light scattering from it. They apply Stokes-Einstein equation to relate diffusion to size (Hydrodynamic diameter). Additionally, DLS offers an ensemble measurement, whereas NTA delivers a particle-by-particle measurement.

The size distribution can be determined using the DLS technique. The distribution is represented by the polydispersity index PDI, whose values are in the range between 0 and 1. Values near 0 represent a monodispersed sample and values near 1 represent polydisperse sample. It can be considered that values below 0.5 have good distribution values.

The positive charge on the external surface of the micro-vesicles can be measured as the zeta potential, which can be determined by electrophoresis. Essentially, an electric filed is applied across a dispersion of the micro-vesicles. Since the velocity is proportional to the magnitude of the zeta potential, the zeta potential can be calculated by detecting the migration rate of particles toward the electrode of opposite charge.

The membrane bilayer of the micro-vesicles according to the present invention is composed as known in the art. Typical micro-vesicles are composed of phospholipids, especially sphingomyelin, phosphatidylcholine and/or cholesterol, but may also include other lipids such as those found in eggs and phosphatidylcholine as long as they are compatible with the bilayer structure.

In particular the membrane bilayer may comprise or be coated with cationic or amphoteric lipids to impart the positive charge onto the outer surface of the micro-vesicle.

By the expression “cationic lipid” it is intended a positively charged amphiphilic molecules constituted by a cationic polar head group (typically amino groups), a hydrophobic domain (alkyl chains or cholesterol), and a linker linking the polar head group with the non-polar tail.

Similarly, by the expression “amphoteric lipid” it is intended a charged amphiphilic molecules constituted by an amphoteric head group (typically a betaine), a hydrophobic domain (alkyl chains or cholesterol), and a linker linking the polar head group with the non-polar tail. Depending on the pH of the milieu, micro-vesicles functionalised with amphoteric lipids can display a net positive charge on their surface.

Amphoteric lipids can be chosen from the list comprising cetyl betaine, myristyl betaine, behenyl betaine, stearyl betaine and lauryl betaine.

Cationic lipids can be chosen from the list comprising laurylpyridinium halide, cetylpyridinium halide, steapyrium halide, stearalkonium halide, lauryl methyl gluceth-10 hydroxypropyldimonium halide, PEG-5 stearyl ammonium halide, didecyldimonium halide, distearoylethyl dimonium halide, lauralkonium halide, lauryl isoquinolinium halide, domiphen halide, tricetylmonium halide, distearyldimonium halide, hydroxypropyl bisstearyldimonium halide and/or dicetyldimonium halide.

Halide can be fluoride, chloride, bromide or iodide. In one embodiment it is chloride.

In a particular embodiment the cationic lipids can be chosen from the list comprising cetylpyridinium chloride, benzalkonium chloride, distearoylethyl dimonium chloride, didecyldimonium chloride, distearyldimonium chloride and/or dicetyldimonium chloride.

The membrane bilayer may comprise other optional components such as bulking agents like mannitol and glycine, thickening agents such as glycerin and/or cetearyl alcohol, metal hydroxides such as sodium hydroxide, preservatives such as potassium sorbate and/or sodium benzoate and alcohols.

The liposomes obtained had an average particle size of 100 to 350 nm, preferably 150 to 300 nm, more preferably 200 nm. The polydispersity was 0.5

Their measured zeta potential was in the range of 2 to 25 mV, preferably 5 to 20 mV, most preferably 10 to 15 mV.

The liposomes according to the invention can be used as such for the delivery of functional active agents to cells, in particular skin cells.

Alternatively, they can be used to prepare exosome-like vectors according to the invention. In particular, the positive charge on the surface of the liposomes derived from their functionalization with a positively charged liposome or from an amphoteric liposome under appropriate pH conditions can be used to functionalize the micro-vesicles through electrostatic interactions with appropriate ligands. These ligands have the function of facilitating cell-to-cell communication and material transfer, in particular in skin cells.

Particularly effective ligands belong to the class of oligopeptides and polypeptides that play a key role in the regulation of physiological processes and have biomimetic sequences similar to those produced and secreted by skin cells such as fibroblasts, keratinocytes, and melanocytes. Specific examples are listed in Table 1:

TABLE 1

| Name | INCI Name | Active site | Effects |
|---|---|---|---|
| EGF | Epidermal Growth | sh-Oligopeptide-1 | Anti-aging, anti-wrinkle, |
| | Factor | (or) rh- | wound healing |
| | | Oligopeptide-1 | |
| aFGF | acidic Fibroblast | sh-Polypeptide-11 | Anti-aging, anti-wrinkle, |
| | Growth Factor | (or) rh- | wound healing, anti-hair |
| | | Polypeptide-11 | loss, hair growth |
| bFGF | basic Fibroblast | sh-Polypeptide-1 | Anti-aging, anti-wrinkle, |
| | Growth Factor | (or) rh- | wound healing, anti-hair |
| | | Polypeptide-1 | loss, hair growth |
| IGF-1 | Insulin-like Growth | sh-Oligopeptide-2 | Anti-aging, anti-wrinkle, |
| | Factor 1 | (or) rh- | wound healing, anti-hair |
| | | Oligopeptide-2 | loss, hair growth |
| IGF-2 | Insulin-like Growth | sh-Polypeptide-31 | Anti-aging, anti-wrinkle, |
| | Factor 2 | (or) rh- | wound healing, anti-hair |
| | | Polypeptide-31 | loss, hair growth |
| AQP | Aquaporin | sh-Polypeptide-64 | Skin morsturizing, anti- |
| | | (or) rh- | aging, anti-wrinkle |
| | | Polypeptide-64 | |
| CCL- | Monocyte | sh-Oligopeptide- | Anti-aging, anti-wrinkle, |
| 2/MCP1 | chemotactic protein 1 | 10 | wound healing |
| COL3A1 | Collagen type III, | Desamido | Anti-aging, anti-wrinkle, |
| | alpha 1 | collagen | wound healing |
| CSF-1 | Colony Stimulating | sh-Polypeptide-73 | Anti-aging, anti-wrinkle, |
| | Factor 1 | | wound healing |
| EPO | Erythroporetin | sh-Polypeptide-72 | Anti-aging, anti-wrinkle, |
| | | | wound healing |
| Flt-3L | Fms-like tyrosine | sh-Polypeptide-26 | Anti-aging, anti-wrinkle, |
| | kinase 3 ligand | (or) rh- | wound healing |
| | | Polypeptide-26 | |
| Fractalkine | Fractalkine | sh-Oligopeptide- | Anti-aging, anti-wrinkle, |
| | | 16 | wound healing |
| hBD3H | human Beta Defensin | sh-Oligopeptide- | Anti-microbial, anti-acne, |
| | 3 Homolog | 33 (or) rh- | anti-atopic dermatitis |
| | | Oligopeptide-33 | |
| HGF | Hepatocyte Growth | sh-Polypeptide-62 | Anti-hair loss, hair growth |
| | Factor | (or) rh- | |
| | | Polypeptide-62 | |
| hGH | human Growth | sh-Polypeptide-7 | Anti-aging, anti-wrinkle, |
| | Hormone | (or) rh- | wound healing |
| | | Polypeptide-7 | |
| HSP70 | Heat Shock Protein | sh-Polypeptide-81 | Anti-aging, anti-wrinkle, |
| | | | wound healing |
| IFN-γ | Interferon gamma | sh-Polypeptide-38 | Anti-aging, anti-wrinkle, |
| | | | wound healing |
| IL-1β | Interleukin-1 beta | sh-Polypeptide-18 | Anti-aging, anti-wrinkle, |
| | | (or) rh- | wound healing |
| | | Polypeptide-18 | |
| IL-2 | Interleukin-2 | sh-Polypeptide-25 | Anti-aging, anti-wrinkle, |
| | | (or) rh- | wound healing |
| | | Polypeptide-25 | |
| IL-6 | Interleukin-6 | sh-Polypeptide-43 | Anti-aging, anti-wrinkle, |
| | | | wound healing |
| IL-8 | Interleukin-8 | sh-Oligopeptide- | Anti-aging, anti-wrinkle, |
| | | 72 | wound healing |
| IL-10 | Interleukin-10 | sh-Polypeptide-6 | Anti-aging, anti-wrinkle, |
| | | (or) rh- | wound healing |
| | | Polypeptide-6 | |
| IP-10 | Interferon gamma- | sh-Oligopeptide- | Anti-aging, anti-wrinkle, |
| | induced Protein 10 | 11 | wound healing |
| KGF-1 | Keratinocyte Growth | sh-Polypeptide-3 | Anti-aging, anti-wrinkle, |
| | Factor 1 | (or) rh- | wound healing, anti-hair |
| | | Polypeptide-3 | loss, hair growth |
| KGF-2 | Keratinocyte Growth | sh-Polypeptide-10 | Anti-aging, anti-wrinkle, |
| | Factor 2 | (or) rh- | wound healing, anti-hair |
| | | Polypeptide-10 | loss, hair growth |
| NGF | Nerve Growth Factor | rh-Polypeptide-14 | Anti-aging, anti-wrinkle, |
| | | | wound healing, anti-hair |
| | | | loss, hair growth |
| Noggin | Noggin | sh-Polypeptide-13 | Anti-hair loss, hair growth |
| | | (or) rh- | |
| | | Polypeptide-13 | |
| PDGF-A | Platelet Derived | sh-Polypeptide-8 | Anti-aging, anti-wrinkle, |
| | Growth Factor A | (or) rh- | wound healing, anti-hair |
| | | Polypeptide-8 | loss, hair growth |
| PDGF-B | Platelet Derived | sh-Polypeptide-59 | Anti-aging, anti-wrinkle, |
| | Growth Factor B | (or) rh- | wound healing, anti-hair |
| | | Polypeptide-59 | loss, hair growth |
| PGF | Placental Growth | sh-Polypeptide-16 | Anti-aging, anti-wrinkle, |
| | Factor | (or) rh- | wound healing |
| | | Polypeptide-16 | |
| SCF | Stem Cell Factor | sh-Polypeptide-4 | Anti-aging, anti-wrinkle, |
| | | (or) rh- | wound healing, anti-hair |
| | | Polypeptide-4 | loss, hair growth |
| SIRT-1 | Sirtuinl | sh-Potypeptide-66 | Anti-aging, anti-wrinkle, |
| | | (or) rh- | wound healing |
| | | Polypeptide-66 | |
| SIRT-2 | Sirtuin2 | sh-Potypeptide-67 | Anti-aging, anti-wrinkle, |
| | | (or) rh- | wound healing |
| | | Polypeptide-67 | |
| SOD-1 | Superoxide Dismutase | sh-Potypeptide-60 | Anti-aging, hair growth, anti- |
| | 1 | (or) rh- | oxidant |
| | | Polypeptide-60 | |
| SOD-3 | Superoxide Dismutase | sh-Polypeptide-51 | Anti-aging, hair growth, anti- |
| | 3 | (or) rh- | oxidant |
| | | Polypeptide-51 | |
| TGF-α | Transforming Growth | sh-Polypqrtide-19 | Anti-aging, anti-wrinkle, |
| | Factor alpha | (or) rh- | wound healing, anti-hair |
| | | Polypeptide-19 | loss, hair growth |
| TGF-β1 | Transforming Growth | sh-Polypeptide-22 | Anti-aging, hair growth, |
| | Factor Beta 1 | (or) rh- | whitening, wound healing |
| | | Polypeptide-22 | |
| TGF-β3 | Transforming Growth | sh-Polypeptide-5 | Anti-aging, hair growth, |
| | Factor Beta 3 | (or) rh- | whitening, wound healing |
| | | Polypeptide-5 | |
| TIMP-1 | Tissue Inhibitor or | sh-Polypeptide-58 | Anti-aging, hair growth, |
| | Metalloproteinase 1 | (or) rh- | wound healing |
| | | Polypeptide-58 | |
| Thymosin-β4 | Thymosin beta 4 | sh-Oligopeptide-4 | Anti-hair loss, hair growth |
| | | (or) rh- | |
| | | Oligopeptide-4 | |
| TNF-α | IhmorNecrosis Factor | sh-Polypeptide-33 | Anti-aging, anti-wrinkle, |
| | alpha | (or) rh- | wound healing, anti-hair |
| | | Polypeptide-33 | loss, hair growth |
| TRX | Thioredoxin | sh-Polypeptide-2 | Anti-aging, hair growth, |
| | | (or) rh- | Anti-oxidant |
| | | Polypeptide-2 | |
| VEGF | Vascular Endothelial | sh-Polypeptide-9 | Anti-aging, anti-wrinkle, |
| | Growth Factor | (or) rh - | wound healing, anti-hair |
| | | Polypeptide-9 | loss, hair growth |
| Laminin | Laminin | Under progress | Anti-aging, hair growth |
| Trade Name | Fullname | INCI Name | Effects |
| Phyto-EGF | EGF from Rice | Transgenic Rice | Anti-aging, anti-wrinkle, |
| | | sh-Oligopeptide-1 | wound healing |
| Phyto-aFGF | aFGF from Potato | Transgenic Potato | Anti-aging, anti-wrinkle, |
| | | sh-Polypeptide-11 | wound healing, anti-hair |
| | | | loss, hair growth |
| Phyto-bFGF | bFGF from Potato | Irasgenic Potato | Anti-aging, anti-wrinkle, |
| | | sh-Polypeptide-1 | wound healing, anti-hair |
| | | | loss, hair growth |
| IntoCell-IGF- | sr-Rainbow Trout | Skin&Hair | Anti-wrinkle, anti-aging, |
| 1 | Oligopeptide-1 sh- | | anti-hair loss |
| | Oligopeptide-2 | | |
| IntoCell- | sr-Rainbow Trout | Skin&Hair | Anti-wrinkle, anti-aging, |
| aFGF | Oligopeptide-1 sh- | | anti-hair loss |
| | Polypeptide 11 | | |
| IntoCell- | sr-Rainbow Trout | Skin&Hair | Anti-wrinkle, anti-aging, |
| bFGF | Oligopeptide-1 sh- | | anti-hair loss |
| | Polypeptide-1 | | |
| IntoCell- | sr-Rainbow Trout | Hair | Anti-hair loss, hair formation |
| Noggin | Oligopeptide-1 sh- | | |
| | Polypeptide-13 | | |
| IntoCell- | sr-Rainbow Trout | Skin&Hair | Anti-wrinkle, anti-aging, |
| PDGF-A | Oligopeptide-1 sh- | | anti-hair loss |
| | Polypeptide-8 | | |
| IntoCell- | sr-Rainbow Trout | Skin&Hair | Anti-wrinkle, anti-aging, |
| VEGF | Oligopeptide-1 sh- | | anti-hair loss |
| | Polypq)tide-9 | | |

In specific examples of the present invention, oligopeptide-1 and oligopeptide-2 were used alone or in combination to create exosome-like vectors.

The exosome-like vectors according to the present invention have a size of 100 to 200 nm and a zeta potential of 2-25 mV, preferably 5 to 20 mV, more preferably 10 to 15 mV.

The exosome-like vectors according to the present invention can be used to deliver to the skin of a patient or cosmetic user a variety of functional agents, for example anti-ageing agents, brightening agents, radiance improving agents, hydrating agents, botox-like agents, anti-sweating agents, sebum regulating agents, anti-wrinkle agents, skin nourishing agents, soothing agents, agents to treat or conceal dark spots, dark circles and/or stretch marks, agents to treat varicous veins, agents for eyelashes/brows growth, exfoliating agents, skin puffiness agents, smoothing agents, tanning agents, skin repairing agents, agents to treat and/or improve signs caused by pollution and/or agents to improve skin barrier and/or any agent fighting cellular senescence and/or an agent addressing skin imperfections including clogged pores, black heads, papules, pustules and any other skin imperfection

Specific agents to be delivered with the exosome-like vectors of the present invention can be selected from the group consisting of melatonin, dimethyl methoxy chromanol, Saccharomyces cervisiae extract, oligopeptide-1, oligopeptide-2, retinol and derivates such as retinal and others, ascorbyl glucoside, vitamin C & derivates in general, bakuchiol, Kombucha, marine enzymes such as from Thermus thermophillus, alpha glucan oligosaccharide, Plantago extract, bifida ferment lysate, plankton extract, micrococcus lyzate, Arabidopsis thaliana extract, bacillus ferment, ethyl ascorbic acid, ascorbic acid, glycyrrhetinic acid and all glycyrrhiza derivates, licorice, tranexamic acid and derivates, barley extract, butterfly bush extract, Physalis angulata extract, Schisandra sphenanthera or chinensis extract, daisy flower extract, peptide(s), algae extract including red algae, phycocyanine, spirullina, any resveratrol and derivates including pterostilbene, asthaxantin and derivates, polyphenols from any source, vitamin E and derivates, flavonoids & prenylated flavonoids, red salvia and any cryptotanshinone containing extract, bisabolol, N-acetylneuraminic acid, any DNA repair enzyme, creatine, Sechium edule extract, sericoside and derivates, green tea extract and more generally all tea species, niacinamide and derivates, any resorcinol and derivate including phenylethyl resorcinol, glutathione and derivates, Nymphaea Caerulea Flower Extract (and)/or Nelumbo Nucifera Flower Extract and more generally any lotus and/or waterlily extract, baicalin, baicalein, tryptophane, quercetin and derivates, any jasmine extract, any microorganism extract and/or ferment and/or supernatant and/or any postbiotic, any antioxidant enzyme like SOD or catalase and others, any anti cell senescence active ingredient, baicaline, baicaleine, azelaic acid and derivates, cysteamine and derivates, carnosine and derivates such as carcinine, all microcirculation boosters like alpha glucosyl hesperidine and others, tangerine peel extract, any peptide of any source and in particular palmitoyl tripeptide-5 and/or acetyl dipeptide-1 cetyl ester, any AHA, Salicylic acid and derivates, adenosine and derivates, astaxanthin and derivates, hyaluronic acid and derivates, saccharide isomerate, ceramides and derivates and precursors, any oil, Ectein, Ginkgo biloba leaf extract and other extracts, scutellaria extract, any Epilobium extract, any Rhododendron extract, Ectoin and derivates, elderberry flower extract, Kigelia africana extract of any part of the plant including fruit, any soy extract, any sweet pea extract, any vegetal peptide.

Additionally, the ligands in Table 1 can also be incorporated into the vesicles as functional (active) agents.

In order to deliver the agent it is first necessary to encapsulate it into micro-vesicle or the exosome-like vector according to the present invention as the case may be. This can be done according to methods known in the art during the process of micro-vesicle manufacturing. Known methods of encapsulation include passive loading encapsulation, which is done by the entrapment of compounds during the process of vesicle formation, and active or remote loading encapsulation, which involves the entrapment of bioactive compounds into intact vesicles.

The micro-vesicles or the exosome-like vectors of the present invention can be formulated according to methods known in the art, in particular in the form of an oil-in-water emulsion, a water-in-oil emulsion, silicone/water emulsions, water/silicone emulsions, a multiple emulsion (water/oil/water or oil/water/oil), a microemulsion, a nanoemulsion, a solution, a suspension, a hydrodispersion, a gel, an ointment, a paste, an aerosol foam, a spray, an aqueous gel, a powder, a foundation, a cream or a mask. In a particular embodiment of the present invention the composition is not in the form of a dressing or patch.

The composition can also be suitable for enteral or parenteral administration, in particular intradermal or subcutaneous administration.

The topical compositions according to the invention may comprise at least one further ingredient commonly found in topical cosmetic and/or medical (e.g. dermatological) compositions chosen from water, oils, which may be chosen in particular from volatile and/or non-volatile, linear or cyclic silicone oils, waxes (such as ozokerite, polyethylene wax, beeswax or carnauba wax), silicone elastomers, nonionic, anionic, cationic and/or amphoteric surfactants, co-surfactant (such as linear fatty alcohols), thickeners, gelling agents, humectants (such as polyols like glycerin), colorants, preservatives, fillers, tensors, sequestrants, perfumes, and mixtures hereof.

These compositions can be in particular in the form of oil-in-water emulsions, water-in-oil emulsions, silicone/water emulsions, water/silicone emulsions, a multiple emulsion (water/oil/water or oil/water/oil) which can optionally be microemulsions or nanoemulsions, or in the form of solutions, suspensions, hydrodispersions, oils, gels, ointments, pastes, aerosol foams or sprays, aqueous gels, powders, or foundations. They can be more or less fluid and have the appearance of creams, emulsions, gels, masks or any other aspect of healthy skin care cosmetics. The person skilled in the art is aware of the procedures, ingredients and forms of common use in the preparation of dermal formulations.

A “gel” is a colloid in which the dispersed phase has combined with the continuous phase to produce a semisolid material, such as jelly.

An “oil” is a composition containing at least 95% wt of a lipophilic substance. Examples of lipophilic substances include but are not limited to naturally occurring and synthetic oils, fats, fatty acids, lecithins, triglycerides and combinations thereof.

A “continuous phase” refers to the liquid in which solids are suspended or droplets of another liquid are dispersed and is sometimes called the external phase. This also refers to the fluid phase of a colloid within which solid or fluid particles are distributed. If the continuous phase is water (or another hydrophilic solvent), water-soluble or hydrophilic drugs will dissolve in the continuous phase (as opposed to being dispersed). In a multiphase formulation (e.g., an emulsion), the discreet phase is suspended or dispersed in the continuous phase. Excipients for topical administration may include anti-microbial compounds, e.g. parabens, antioxidants, e.g. sodium ascorbyl acetate and alpha-tocopherol, stabilizers, e.g. sorbitol, and/or emulsifying agents to produce a stable emulsion with both a hydrophilic and a hydrophobic phase.

“Diluents” may be included in the formulations to dissolve, disperse or otherwise incorporate the carrier. Examples of diluents include, but are not limited to, water, buffered aqueous solutions, organic hydrophilic diluents, such as monovalent alcohols, and low molecular weight glycols and polyols (e.g. propylene glycol, polypropylene glycol, glycerol, butylene glycol).

Appropriate excipients are selected based on the type of formulation. Standard excipients include gelatin, casein, lecithin, gum acacia, cholesterol, tragacanth, stearic acid, benzalkonium chloride, calcium stearate, glyceryl monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters, polyethylene glycols, polyoxyethylene stearates, colloidol silicon dioxide, phosphates, sodium dodecyl sulfate, carboxymethylcellulose calcium, carboxymethylcellulose sodium, methylcellulose, hydroxyethylcellulose, hydroxypropylcellulose, hydroxypropylmethycellulose phthalate, noncrystalline cellulose, magnesium aluminum silicate, triethanolamine, polyvinyl alcohol, polyvinylpyrrolidone, sugars, and starches.

An emulsion is a preparation of one liquid distributed in small globules throughout the body of a second liquid. The dispersed liquid is the discontinuous phase, and the dispersion medium is the continuous phase. When oil is the dispersed liquid and an aqueous solution is the continuous phase, it is known as an oil-in-water emulsion, whereas when water or aqueous solution is the dispersed phase and oil or oleaginous substance is the continuous phase, it is known as a water-in-oil emulsion. The oil phase may consist at least in part of a propellant, such as an HFA propellant. Either or both of the oil phase and the aqueous phase may contain one or more surfactants, emulsifiers, emulsion stabilizers, buffers, and other excipients. Preferred excipients include surfactants, especially non-ionic surfactants; emulsifying agents, especially emulsifying waxes; and liquid non-volatile non-aqueous materials, particularly glycols such as propylene glycol. The oil phase may contain other oily pharmaceutically approved excipients. For example, materials such as hydroxylated castor oil or sesame oil may be used in the oil phase as surfactants or emulsifiers.

“Emollients” are an externally applied agent that softens or soothes skin and are generally known in the art and listed in compendia, such as the “Handbook of Pharmaceutical Excipients”, 4th Ed., Pharmaceutical Press, 2003. These include, without limitation, almond oil, castor oil, ceratonia extract, cetostearoyl alcohol, cetyl alcohol, cetyl esters wax, cholesterol, cottonseed oil, cyclomethicone, ethylene glycol palmitostearate, glycerin, glycerin monostearate, glyceryl monooleate, isopropyl myristate, isopropyl palmitate, lanolin, lecithin, light mineral oil, medium-chain triglycerides, mineral oil and lanolin alcohols, petrolatum, petrolatum and lanolin alcohols, soybean oil, starch, stearyl alcohol, sunflower oil, xylitol and combinations thereof. In one embodiment, the emollients are ethylhexylstearate and ethylhexyl palmitate.

“Surfactants” are surface-active agents that lower surface tension and thereby increase the emulsifying, foaming, dispersing, spreading and wetting properties of a product. Suitable non-ionic surfactants include emulsifying wax, glyceryl monooleate, polyoxyethylene alkyl ethers, polyoxyethylene castor oil derivatives, polysorbate, sorbitan esters, benzyl alcohol, benzyl benzoate, cyclodextrins, glycerin monostearate, poloxamer, povidone and combinations thereof. In one embodiment, the non-ionic surfactant is stearyl alcohol.

“Emulsifiers” are surface active substances which promote the suspension of one liquid in another and promote the formation of a stable mixture, or emulsion, of oil and water. Common emulsifiers are: metallic soaps, certain animal and vegetable oils, and various polar compounds. Suitable emulsifiers include acacia, anionic emulsifying wax, calcium stearate, carbomers, cetostearyl alcohol, cetyl alcohol, cholesterol, diethanolamine, ethylene glycol palmitostearate, glycerin monostearate, glyceryl monooleate, hydroxpropyl cellulose, hypromellose, lanolin, hydrous, lanolin alcohols, lecithin, medium-chain triglycerides, methylcellulose, mineral oil and lanolin alcohols, monobasic sodium phosphate, monoethanolamine, nonionic emulsifying wax, oleic acid, poloxamer, poloxamers, polyoxyethylene alkyl ethers, polyoxyethylene castor oil derivatives, polyoxyethylene sorbitan fatty acid esters, polyoxyethylene stearates, propylene glycol alginate, self-emulsifying glyceryl monostearate, sodium citrate dehydrate, sodium lauryl sulfate, sorbitan esters, stearic acid, sunflower oil, tragacanth, triethanolamine, xanthan gum and combinations thereof. In one embodiment, the emulsifier is glycerol stearate.

A “lotion” is a low- to medium-viscosity liquid formulation. A lotion can contain finely powdered substances that are in soluble in the dispersion medium through the use of suspending agents and dispersing agents. Alternatively, lotions can have as the dispersed phase liquid substances that are immiscible with the vehicle and are usually dispersed by means of emulsifying agents or other suitable stabilizers. In one embodiment, the lotion is in the form of an emulsion having a viscosity of between 100 and 1000 centistokes. The fluidity of lotions permits rapid and uniform application over a wide surface area. Lotions are typically intended to dry on the skin leaving a thin coat of their medicinal components on the skin's surface.

A “cream” is a viscous liquid or semi-solid emulsion of either the “oil-in-water” or “water-in-oil type”. Creams may contain emulsifying agents and/or other stabilizing agents. In one embodiment the formulation is in the form of a cream having a viscosity of greater than 1000 centistokes, typically in the range of 20,000-50,000 centistokes. Creams are often time preferred over ointments as they are generally easier to spread and easier to remove.

An “ointment” is a semisolid preparation containing an ointment base and optionally one or more active agents. Examples of suitable ointment bases include hydrocarbon bases (e.g., petrolatum, white petrolatum, yellow ointment, and mineral oil); absorption bases (hydrophilic petrolatum, anhydrous lanolin, lanolin, and cold cream); water-removable bases (e.g., hydrophilic ointment), and water-soluble bases (e.g., polyethylene glycol ointments). Pastes typically differ from ointments in that they contain a larger percentage of solids. Pastes are typically more absorptive and less greasy that ointments prepared with the same components.

A “gel” is a semisolid system containing dispersions of small or large molecules in a liquid vehicle that is rendered semisolid by the action of a thickening agent or polymeric material dissolved or suspended in the liquid vehicle. The liquid may include a lipophilic component, an aqueous component or both. Some emulsions may be gels or otherwise include a gel component. Some gels, however, are not emulsions because they do not contain a homogenized blend of immiscible components. Suitable gelling agents include, but are not limited to, modified celluloses, such as hydroxypropyl cellulose and hydroxyethyl cellulose; Carbopol homopolymers and copolymers; and combinations thereof. Suitable solvents in the liquid vehicle include, but are not limited to, diglycol monoethyl ether; alklene glycols, such as propylene glycol; dimethyl isosorbide; alcohols, such as isopropyl alcohol and ethanol. The solvents are typically selected for their ability to dissolve the drug. Other additives, which improve the skin feel and/or emolliency of the formulation, may also be incorporated. Examples of such additives include, but are not limited, isopropyl myristate, ethyl acetate, C12-C15 alkyl benzoates, mineral oil, squalane, cyclomethicone, capric/caprylic triglycerides, and combinations thereof.

“Foams” consist of an emulsion in combination with a gaseous propellant. The gaseous propellant consists primarily of hydrofluoroalkanes (HFAs). Suitable propellants include HFAs such as 1,1,1,2-tetrafluoroethane (HFA 134a) and 1,1,1,2,3,3,3-heptafluoropropane (HFA 227), but mixtures and admixtures of these and other HFAs that are currently approved or may become approved for medical use are suitable. In one embodiment the propellants are not hydrocarbon propellant gases which can produce flammable or explosive vapors during spraying. Furthermore, exemplary compositions according to the present invention contain no volatile alcohols, which can produce flammable or explosive vapors during use.

Buffers are used to control pH of a composition. In one embodiment, the buffers buffer the composition from a pH of about 4 to a pH of about 7.5, or from a pH of about 4 to a pH of about 7, or from a pH of about 5 to a pH of about 7. In a preferred embodiment, the buffer is based on triethanolamine, citric acid, sodium hydroxyde, disodium phosphate and potassium phosphate.

Preservatives can be used to prevent the growth of fungi and microorganisms. Suitable antifungal and antimicrobial agents include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, potassium sorbate, caprylyl alcohol, 1,2-hexandiol, and thimerosal.

Antioxidants can be used in the compositions of the present invention. Examples of antioxidants are tocopherol, hydroxyacetophenone, dimethylmethoxy chromanol and phenethyl alcohol.

Viscosity modifiers can additionally be present, such as potassium chloride and natural gums such as xanthan gum, gum arabic, gum tragacanth, carrageenan, chitosan, guar gum, konjac gum, sclerotium gum, dextrins and starch.

Chelating agents may additionally be present, such as sodium glucuronate.

Fragrances may be added to the compositions of the invention.

Penetration enhancers are frequently used to promote transdermal delivery of drugs across the skin, in particular across the stratum corneum. The more commonly used enhancers include urea, (carbonyldiamide), imidurea, N, N-diethylformamide, N-methyl-2-pyrrolidine, 1-dodecal-azacyclopheptane-2-one, calcium thioglycate, 2-pyyrolidine, N,N-diethyl-m-toluamide, oleic acid and its ester derivatives, such as methyl, ethyl, propyl, isopropyl, butyl, vinyl and glycerylmonooleate, sorbitan esters, such as sorbitan monolaurate and sorbitan monooleate, other fatty acid esters such as isopropyl laurate, isopropyl myristate, isopropyl palmitate, diisopropyl adipate, propylene glycol monolaurate, propylene glycol monooleatea and non-ionic detergents such as BRIJ® 76 (stearyl poly(10 oxyethylene ether), BRIJ® 78 (stearyl poly(20)oxyethylene ether), BRIJ® 96 (oleyl poly(10)oxyethylene ether), and BRIJ® 721 (stearyl poly (21) oxyethylene ether) (ICI Americas Inc. Corp.).

Emulsion stabilizers, surfactants and emulsifying agents may additionally be present, such as sorbitan isostearate, hydroxyethyl acrylate/sodium acryloxydiethyl taurate copolymer and sorbitan isostearate.

Humectants are crucial in skincare by providing essential skin hydration and maintaining the skin's moisture balance. Exemplary humectants are Glycerin, Hyaluronic acid, Aloe vera, Honey, Hydrolyzed proteins, Panthenol, Allantoin, Seaweed & algae, Sodium PCA (sodium pyrrolidone carboxylic acid), Saccharide Isomerate, Sugar Alcohols (e.g. sorbitol), Hydroxy acids (e.g. lactic acid and gluconolactone), Agave nectar, Betaine, Chitosan, Trehalose, Beta-glucan, Topical collagen, Galactoarabinan and propanediol

Solvents used in the compositions of the present invention may include water, alcohols such as ethanol and isopropyl alcohol, alkanes such as C15-19 Alkane, silicone oils and mineral oils.

Skin conditioning agents may be added to the compositions of the present invention, for example caprylic/capric triglyceride, alpha-glucan triglycerides, palmitoyl tripeptide-5, glycerine, diglucosyl gallic acid, panthenol, pantolactone, sodium stearoyl glutamate, lactic acid, hydrolysed hyaluronic acid, sodium hyaluronate and maltodextrin.

Further natural extracts may be present in compositions of the present invention, for example Saccharomyces/Xylinum/Black Tea Ferment, Bellis perennis (Daisy) Flower Extract, Thermus thermophillus Ferment and Sphingomonas Ferment Extract.

After formulation, the product is filled into an appropriate dispenser and shipped to the end user. Examples of final container may include a pump bottle, squeeze bottle, jar, tube or vial.

The compositions according to the invention may additionally comprise one or more UVA filter(s), one or more UVB filter(s) and/or one or more UVA/UVB filter(s).

Sunscreen filters that may be present in a composition of the present invention may be:
A. Screens, which use only inorganic compounds (mainly zinc oxide and/or titanium dioxide) as active ingredients. These ingredients primarily work by absorbing UV rays but also through reflection and refraction; and
B. Chemical sunscreens, which use organic molecules as active ingredients; and/or
C. Mixtures thereof.

By way of illustration of organic UV filters and in a non-limiting manner, mention may be made of:
anthranilates, in particular menthyl anthranilate;
benzophenones, in particular 1-benzophenone, 3-benzophenone or oxybenzone, 5-benzophenone, 6-benzophenone, 8-benzophenone, 9-benzophenone, 12-benzophenone, and exemplarily 2-benzophenone (Oxybenzone), or 4-benzophenone (Uvinul MS40® available from B.A.S.F.);
benzylidenecamphor, in particular 3-benzylidenecamphor, benzylidenecamphorsulphonic acid, benzalkoniumcamphor methosulfate, polyacrylamidomethylbenzylidenecamphor, terephthalylidenecamphorsulphonic acid, and exemplarily 4-methylbenzylidenecamphor (Eusolex 6300® available from Merck);
benzimidazoles, in particular benzimidazilate (Neo Heliopan AP® available from Haarmann and Reimer), or phenylbenzimidazole sulfonic acid (PARSOL HS® available from DSM);
benzotriazoles, in particular drometrizole trisiloxane, or methylene bis-benzotriazolyltetramethylbutylphenol (Tinosorb M® available from Ciba);
cinnamates, in particular cinoxate, DEA methoxycinnamate, diisopropyl methylcinnamate, dimethoxycinnamate glyceryl ethylhexanoate, isopropyl methoxycinnamate, isoamyl cinnamate, Kaempferia galanga root extract (TEGO GALANGA from EVONIK containing 98% ethyl-p-methoxycinnamate) and exemplarily octylmethoxycinnamate (Parsol MCX® available from Hoffmann La Roche);
diphenylacrylates, in particular ethocrylene (Uvinul N35® available from B.A.S.F.), or octocrylene (Uvinul 539® available from B.A.S.F.) or Ethylhexyl methoxycrylene (SOLASTAY available from HALLSTAR);
dibenzoylmethanes, in particular butyl methoxydibenzoylmethane (Parsol 1789®);
imidazolines, in particular ethylhexyl dimethoxybenzylidenedioxoimidazoline;
PABA, in particular ethyl dihydroxypropyl PABA, ethylhexyldimethyl PABA, glyceryl PABA, PABA, PEG-25 PABA, or ethyl PABA (benzocaine);
triazines, in particular anisotriazine (Tinosorb S® available from Ciba) or diethylhexylbutamido-triazone (Uvasorb HEB® available from 3V Sigma), ethylhexyltriazone (Uvinul T150® available from B. A. S. F.), Tris-Biphenyl Triazine (Tinosorb A2B available from BASF), benzoates, in particular N-hexyl 2-(4-diethylamino-2-hydroxybenzoyl)benzoate (Uvinul A+ available from BASF) or as a mixture with octyl methoxycinnamate (Uvinul A+B available from BASF),
benzalmalonates, in particular Polysilicone-15 (Parsol SLX available from DSM),
benzoxazoles, in particular 2,4-bis[4-[5-(1.1-dimethylpropyl)benzoxazol-2-yl]phenylimino]-6-[(2-ethylhexyl)imino]-1.3,5-triazine, (Uvasorb K2A available from Sigma 3 V);
salicylates, in particular dipropylene glycol salicyclate, ethylhexyl salicylate, homosalate, butyloctyl salicylate (HALLBRITE BHB available from HALLSTAR or TEA salicylate;
Phenylbenzimidazole sulfonic acid (eg Parsol HS), Octocrylene (eg Parsol 340), Ethylhexyl Methoxycinnamate (eg Parsol MCX), Methylene Bis-Benzotriazolyl Tetramethylbutylphenol (eg Tinosorb M), Bis-Ethylhexyloxyphenol Methoxyphenyl Triazine (eg Tinosorb S), Tris-Biphenyl Triazine (eg Tinosorb A2B), Ethylhexyl Triazone (eg UVINUL T150) and Diethylhexyl Butamido Triazone (eg UVASORB HEB), Diethylamino Hydroxybenzoyl Hexyl Benzoate (eg Uvinul A PLUS), Butyl Methoxydibenzoylmethane (eg Parsol 1789), Polysilicone-15 (eg Parsol SLX), Benzophenone-4 (eg Uvinul MS 40), Benzophenone-3, Ethylhexyl Salicylate (eg Parsol EHS), Homosalate;
and/or mixtures thereof.

In a preferred embodiment the sunlight protecting component of the cosmetic compositions according to the invention comprises a mixture of BIS-ETHYLHEXYLOXYPHENOL METHOXYPHENYL TRIAZINE (BEMT) and DIETHYLHEXYL BUTAMIDO TRIAZONE (DHBT).

In a further preferred embodiment, it may additionally comprise at least two of ETHYLHEXYL TRIAZONE (EHT), HOMOSALATE and/or BUTYL METHOXYDIBENZOYLMETHANE.

In a further preferred embodiment it may further additionally comprise PHENYLBENZIMIDAZOLE SULFONIC ACID (also together with OCTYL SALICYLATE) and/or DIETHYLAMINO HYDROXYBENZOYL HEXYL BENZOATE.

The inorganic UV screening agents used are metal oxide particles having an average elementary particle size between about 5 and about 600 nm. In particular embodiments the particle size is 15, 30, 50, 100, 150, 200, 250, 300, 350, 400, 450, 500, 550 nm. They may be chosen in particular from titanium oxide, zinc oxide, iron oxide, zirconium oxide, cerium oxide, and mixtures thereof.

The titanium oxides may be in a crystallized form of the rutile and/or anatase type, and/or in an amorphous or substantially amorphous form. Such coated or uncoated metal oxide pigments are described in particular in patent application EP-A-0,518,773. As commercial pigments, mention may be made of the products sold by the companies Kemira, Tayca, Merck and Degussa.

The metal oxide pigments may be coated or uncoated.

The coated pigments are pigments which have undergone one or more surface treatments of chemical, electronic, mechanochemical and/or mechanical nature with compounds such as amino acids, beeswax, fatty acids, fatty alcohols, anionic surfactants, lecithin, sodium, potassium, zinc, iron or aluminum salts of fatty acids, metal (titanium or aluminum) alkoxides, polyethylene, silicones, proteins (collagen, elastin), alkanolamines, silicon oxides, metal oxides or hexametaphosphate of fatty acids.

Examples of coated titanium oxide pigments are:
silica (SUNVEIL from IKEDA), silica and iron oxide (SUNVEIL F from IKEDA), silica and polyglyceryl-10 stearate (COSMESERVE WP-40W from IWASE COSFA), silica and alumina (MICROTITANIUM DIOXIDE MT500 SA and MICROTITANIUM DIOXIDE MT 100 SA from TAYCA, TIOVEIL from TIOXIDE), alumina (TIPAQUE TTO-55 (B) and TIPAQUE TTO-55 (A) from ISHIHARA, and UVT 14/4 from KEMIRA), Rutile TiO2 treated with alumina and glycerol-coated silica (UV TITAN M212 from KEMIRA), Rutile TiO2 treated with alumina and dimethicone (UV TITAN M195 from KEMIRA), Alumina and aluminum stearate (Microtitanium Dioxide MT 100 T, MT 100 TV, MT 100 TX, MT 100 Z, MT-01 from TAYCA, Solaveil CT-10 W and Solaveil CT 100 from UNIQEMA and Eusolex T-AVO from MERCK), silica, alumina and alginic acid (MT-100 AQ from TAYCA), alumina and aluminum laurate (TAYCA MICROTITANIUM DIOXIDE MT 100 S), alumina, methicone and polyhydroxystearic acid (INP60T7 from KOBO), iron oxide and iron stearate (TAYCA MICROTITANIUM DIOXIDE MT 100 F), zinc oxide and zinc stearate (TAYCA BR 351), silica and alumina and treated with silicone (MICROTITANIUM DIOXIDE MT 600SAS, MICROTITANIUM DIOXIDE MT 500 SAS or MICROTITANIUM DIOXIDE MT 100 SAS from TAYCA), silica, alumina, aluminum stearate and treated with a silicone (STT-30-DS from TITAN KOGYO), alumina and treated with a silicone (TIPAQUE TTO-55 (S) from ISHIHARA, or UV TITAN M 262 from KEMIRA), triethanolamine (STT-65-S from TITAN KOGYO);
stearic acid (TIPAQUE TTO-55 (C) from ISHIHARA, strearic acid (and) aluminium hydroxide (MPT-186 SAKAI) or isostearic acid (MioShade T-Plus Natural Mioshy))
sodium hexametaphosphate (MICROTITANIUM DIOXIDE MT 150 W TAYCA)
TiO2 treated with octyl trimethyl silane (T 805 by the company DEGUSSA SILICES);
TiO2 treated with a polydimethylsiloxane (70250 UF TiO2S13 framework per framework);
anatase/rutile TiO2 treated with a polydimethylhydrogenosiloxane (MICRO TITANIUM DIOXIDE USP GRADE HYDROPHOBIC by COLOR TECHNIQUES),
Rutile TiO2 treated with alumina, stearic acid (UV TITAN M160 by KEMIRA),
TiO2 treated with alumina hydroxide, stearic acid and triethoxycaprylylsilane (ALT-T-400 by MAPRECOS) or alumina hydroxide, stearic acid (and) dimethicone (Mioshade T-clear Mioshi), or alumina and hydrogen dimethicone (TTO-MS4 KOBO): or
manganese-doped TiO2 (Croda OPT 1-PW);
and/or their mixtures.

Examples of uncoated titanium oxide pigments are:
MICROTITANIUM DIOXIDE MT 500 B or MICROTITANIUM DIOXIDE MT600 B by TAYCA;
P 25 by DEGUSSA;
Transparent titanium oxide PW by WACKER;
UFTR by MIYOSHI KASEI;
ITS by TOMEN;
TIOVEIL AQ by the company TIOXIDE
and/or their mixtures.

Examples of uncoated zinc oxide pigments are:
Z-cote by Sunsmart;
Nanox by Elementis;
Nanogard WCD 2025 by Nanophase Technologies
and/or their mixtures.

Coated zinc oxide pigments are, for example:
zinc oxide CS-5 by Toshibi (ZnO coated with polymethylhydrogenesiloxane);
Nanogard Zinc Oxide FN by Nanophase Technologies (40% dispersion in Finsolv TN, C12-C15 alcohol benzoate);
DAITOPERSION ZN-30 and DAITOPERSION Zn-50 by the company Daito (dispersions in cyclopolymethylsiloxane/oxyethylenated polydimethylsiloxane, containing 30% or 50% of zinc nano-oxides coated with silica and polymethylhydrogenenesiloxane);
NFD Ultrafine ZnO by Daikin (ZnO coated with perfluoroalkyl phosphate and perfluoroalkylethyl copolymer in dispersion in cyclopentasiloxane);
SPD-Z1 by Shin-Etsu (ZnO coated with silicone grafted acrylic polymer, dispersed in cyclodimethylsiloxane);
Escalol Z100 by the company ISP (ZnO treated with alumina and dispersed in the mixture of ethylhexyl methoxycinnamate/PVP-hexadecene/methicone copolymer);
Fuji ZnO-SMS-10 by Fuji Pigment (ZnO coated silica and polymethylsilsesquioxane);
Nanox Gel TN by the company Elementis (ZnO dispersed at 55% in C12-C15 alcohol benzoate with hydroxystearic acid polycondensate);
OTS-5 MZ-500 by DAITO (ZnO dispersed in triethoxycaprylylsilane);
natural coatings for example jojoba esters (ZnO-750-NJE7 KOBO) Hydrogenated Olive Oil Stearyl Esters (MZO-35-NOE7 KOBO)
and/or their mixtures.

The uncoated cerium oxide pigments may be, for example, those sold under the name Colloidal Cerium Oxide by the company Rhone Poulenc.

In one embodiment, the zinc and titanium filters are coated, for example they are Zinc Oxide (and) Triethoxycaprylylsilane (INCI) and/or Titanium dioxide (And) Aluminum hydroxide (And) Aluminum stearate (INCI). In another embodiment they are Zinc Oxide (and) Triethoxycaprylylsilane (and) Ethyl Ferulate (INCI), Zinc Oxide (And) Stearoyl Glutamic Acid (INCI) or Titanium Dioxide (and) Isostearic Acid (INCI)

If they are both present the ratio depends on the target SPF: for example, for SPF 50 they are present at a ratio ZnO:TiO2 between 3:1 and 4:1.

The concentration of the micro-vesicles or the exosome-like vectors in the formulation varies according to the formulation. For example, in a daily cream it can range between about 0.1% and about 10%, in a serum between about 0.1% and about 10%, in a mask between 0.1% and about 10%, in ampoules between about 0.1% and about 10%.

Further within the scope of the present invention are cosmetic and medical methods for the treatment of aging and senescence using the micro-vesicles and exosome-like vectors of the present invention.

### EXAMPLES

The following examples illustrate particular embodiments of the invention.

### Example 1: Positively Charged Liposome

Liposomes falling under the scope of the present invention were prepared using methods known in the art and had the following final composition:

| Ingredient | CAS | EC | % (w/w) |
|---|---|---|---|
| AQUA | 7732-18-5 | 231-791-2 | Q.S. 100 |
| PHOSPHATIDYLCHOLINE | 8002-43-5 | 232-307-2 | 1.00-20.00 |
| CAPRYLIC/CAPRIC | 73398-61-5 | 277-452-2 | 1.00-10.00 |
| TRIGLYCERIDE | | | |
| MANNITOL | 69-65-8 | 200-711-8 | 1.00-10.00 |
| GLYCERIN | 56-81-5 | 200-289-5 | 1.-6.00 |
| CITRIC ACID | 77-92-9 | 201-069-1 | 0.50-5.00 |
| POLYGLYCERYL-10 | 34406-66-1 | — | 0.1-5.00 |
| LAURATE | | | |
| MELATONIN | 73-31-4 | 200-797-7 | 0.05-5 |
| POTASSIUM SORBATE | 24634-61-5 | 246-376-1 | 0.10-1.00 |
| SODIUM BENZOATE | 532-32-1 | 208-534-8 | 0.1-1.00 |
| CETEARYL ALCOHOL | 67762-27-0 | 267-008-6 | 0.10-1.00 |
| DISTEAROYLETHYL | 67846-68-8 | 267-382-0 | 0.005-0.5 |
| DIMONIUM | | | |
| CHLORIDE | | | |

(source: Nanovex Biotechnologies S.L., Parque Tecnologico de Asturias, CEEI 33428 Llanera (Asturias), Spain)

### Example 2: Exosome-Like Vector

An exosome-like vector falling under the scope of the present invention were prepared methods known in the art and had the following final composition:

| Ingredient | CAS | EC | % (w/w) |
|---|---|---|---|
| AQUA | 7732-18-5 | 231-791-2 | Q.S. 100 |
| PHOSPHATIDYL- | 8002-43-5 | 232-307-2 | 1.00-20.00 |
| CHOLINE | | | |
| CAPRYLIC/CAPRIC | 73398-61-5 | 277-452-2 | 1.00-10.00 |
| TRIGLYCERIDE | | | |
| MANNITOL | 69-65-8 | 200-711-8 | 1.00-10.00 |
| GLYCERIN | 56-81-5 | 200-289-5 | 1.-6.00 |
| CITRIC ACID | 77-92-9 | 201-069-1 | 0.50-5.00 |
| POLYGLYCERYL-10 | 34406-66-1 | — | 0.1-5.00 |
| LAURATE | | | |
| MELATONIN | 73-31-4 | 200-797-7 | 0.05-5 |
| POTASSIUM SORBATE | 24634-61-5 | 246-376-1 | 0.10-1.00 |
| SODIUM BENZOATE | 532-32-1 | 208-534-8 | 0.1-1.00 |
| CETEARYL ALCOHOL | 67762-27-0 | 267-008-6 | 0.10-1.00 |
| DISTEAROYLETHYL | 67846-68-8 | 267-382-0 | 0.005-0.5 |
| DIMONIUM CHLORIDE | | | |
| OLIGOPEPTIDE-2 | — | — | 0.001-0.9 ppm |
| SODIUM CHLORIDE | 7647-14-5 | 231-598-3 | 0.001-0.9 ppm |
| DIPOTASSIUM | 7758-11-4 | 231-834-5 | 0.0001-0.8 ppm |
| PHOSPHATE | | | |
| OLIGOPEPTIDE-1 | — | — | 0.000001-0.10 ppm |
| POTASSIUM | 7778-77-0 | 231-913-4 | 0.000001-0.10 ppm |
| PHOSPHATE | | | |

The terms and expressions that have been employed are used as terms of description and not of limitation, and there is no intention in the use of such terms and expressions of excluding any equivalents of the features shown and described or portions thereof, but it is recognized that various modifications are possible within the scope of the embodiments of the present invention. Thus, it should be understood that although the present invention has been specifically disclosed by specific embodiments and optional features, modification and variation of the concepts herein disclosed may be resorted to by those of ordinary skill in the art, and that such modifications and variations are considered to be within the scope of embodiments of the present invention.

1. A liposome with positive or amphoteric charges on its surface, wherein the liposome has a zeta potential of 2 to 25 mV, preferably 5 to 20 mV, more preferably 10 to 15 mV.

2. A liposome according to claim 1, wherein the charges are chosen from a list consisting of cetyl betaine, myristyl betaine, behenyl betaine, stearyl betaine, lauryl betaine, laurylpyridinium halide, cetylpyridinium halide, steapyrium halide, stearalkonium halide, lauryl methyl gluceth-10 hydroxypropyldimonium halide, PEG-5 stearyl ammonium halide, didecyldimonium halide, distearoylethyl dimonium halide, lauralkonium halide, lauryl isoquinolinium halide, domiphen halide, tricetylmonium halide, distearyldimonium halide, hydroxypropyl bisstearyldimonium halide and/or dicetyldimonium halide.

3. A liposome according to claim 2, wherein the charges are positive, in particular cetylpyridinium chloride, benzalkonium chloride, distearoylethyl dimonium chloride, didecyldimonium chloride, distearyldimonium chloride and/or dicetyldimonium chloride.

4. A liposome according to claim 1, wherein the liposome comprises phospholipids, preferably phosphatidyl choline.

5. A liposome according to claim 1, wherein the liposome has a size of 100 to 350 nm, preferably 150 to 300 nm.

6. A liposome according to according to claim 1, further comprising:

| Ingredient | CAS | EC | % (w/w) |
|---|---|---|---|
| AQUA | 7732-18-5 | 231-791-2 | Q.S. 100 |
| PHOSPHATIDYLCHOLINE | 8002-43-5 | 232-307-2 | 1.00-20.00 |
| CAPRYLIC/CAPRIC | 73398-61-5 | 277-452-2 | 1.00-10.00 |
| TRIGLYCERIDE | | | |
| MANNITOL | 69-65-8 | 200-711-8 | 1.00-10.00 |
| GLYCERIN | 56-81-5 | 200-289-5 | 1.- 6.00 |
| CITRIC ACID | 77-92-9 | 201-069-1 | 0.50-5.00 |
| POLYGLYCERYL-10 | 34406-66-1 | — | 0.1-5.00 |
| LAURATE | | | |
| MELATONIN | 73-31-4 | 200-797-7 | 0.05-5 |
| POTASSIUM SORBATE | 24634-61-5 | 246-376-1 | 0.10-1.00 |
| SODIUM BENZOATE | 532-32-1 | 208-534-8 | 0.1-1.00 |
| CETEARYL ALCOHOL | 67762-27-0 | 267-008-6 | 0.10-1.00 |
| DISTEAROYLETHYL | 67846-68-8 | 267-382-0 | 0.005-0.5 |
| DIMONIUM | | | |
| CHLORIDE | | | |

7. An exosome-like vector comprising a liposome according to claim 1, further functionalized with peptides, including oligopeptide-1, oligopeptide-2 or combinations thereof via electrostatic binding with the positive charges.

8. An exosome-like vector according to claim 7, wherein the exosome has a size of 100 to 200 nm.

9. An exosome-like vector according to claim 7, having a zeta potential of 2-25 mV.

10. An exosome-like vector according to claim 7, comprising:

| Ingredient | CAS | EC | % (w/w) |
|---|---|---|---|
| AQUA | 7732-18-5 | 231-791-2 | Q S 100 |
| PHOSPHATIDYL- | 8002-43-5 | 232-307-2 | 1.00-20.00 |
| CHOLINE | | | |
| CAPRYLIC/CAPRIC | 73398-61-5 | 277-452-2 | 1.00-10.00 |
| TRIGLYCERIDE | | | |
| MANNITOL | 69-65-8 | 200-711-8 | 1.00-10.00 |
| GLYCERIN | 56-81-5 | 200-289-5 | 1.-6.00 |
| CITRIC ACID | 77-92-9 | 201-069-1 | 0.50-5.00 |
| POLYGLYCERYL-10 | 34406-66-1 | — | 0.1-5.00 |
| LAURATE | | | |
| MELATONIN | 73-31-4 | 200-797-7 | 0.05-5 |
| POTASSIUM SORBATE | 24634-61-5 | 246-376-1 | 0.10-1.00 |
| SODIUM BENZOATE | 532-32-1 | 208-534-8 | 0.1-1.00 |
| CETEARYL ALCOHOL | 67762-27-0 | 267-008-6 | 0.10-1.00 |
| DISTEAROYLETHYL | 67846-68-8 | 267-382-0 | 0.005-0.5 |
| DIMONIUM CHLORIDE | | | |
| OLIGOPEPTIDE-2 | — | — | 0.001-0.9 ppm |
| SODIUM CHLORIDE | 7647-14-5 | 231-598-3 | 0.001-0.9 ppm |
| DIPOTASSIUM | 7758-11-4 | 231-834-5 | 0.0001-0.8 ppm |
| PHOSPHATE | | | |
| OLIGOPEPTIDE-1 | — | — | 0.000001-0.10 ppm |
| POTASSIUM | 7778-77-0 | 231-913-4 | 0.000001-0.10 ppm |
| PHOSPHATE | | | |

11. An exosome-like vector according claim 7 further comprising a pharmacologically or cosmetically active agent.

12. An exosome-like vector according to claim 11, wherein the active agent is located inside the exosome.

13. A liposome according to claim 11, wherein the active agent is an anti-ageing agent, a brightening agent, a radiance improving agent, a hydrating agent, a botox-like agent, an anti-sweating agent, a sebum-regulating agent, an anti-wrinkle agent, a skin nourishing agent, a soothing agent, an agents to treat or conceal dark spots, dark circles and/or stretch marks, an agent to treat varicous veins, an agent for eyelashes/brows growth, an exfoliating agent, a skin puffiness agent, a smoothing agent, a tanning agent, a skin repairing agent, an agent to treat and/or improve signs caused by pollution and/or an agent to improve a barrier function of the skin and/or and/or any agent fighting cellular senescence and/or an agent addressing skin imperfections including clogged pores, black heads, papules, pustules and any other skin imperfection.

14. A cationic micro-vesicle or an exosome-like vector according to claim 12, wherein the active agent comprises an agent selected from the group consisting of Melatonin, dimethyl methoxy chromanol, Saccharomyces cervisiae extract, oligopeptide-1, oligopeptide-2, retinol and derivates such as retinal and others, ascorbyl glucoside, vitamin C & derivates in general, niacinamide, bakuchiol, Kombucha, marine enzymes such as from Thermus thermophillus, alpha glucan oligosaccharide, Plantago extract, bifida ferment lysate, plankton extract, micrococcus lyzate, Arabidopsis thaliana extract, bacillus ferment, ethyl ascorbic acid, ascorbic acid, glycyrrhetinic acid and all glycyrrhiza derivates, licorice, tranexamic acid and derivates, barley extract, butterfly bush extract, Physalis angulata extract, Schisandra sphenanthera or chinensis extract, daisy flower extract, peptide(s), algae extract including red algae, phycocyanine, spirullina, any resveratrol and derivates including pterostilbene, asthaxantin and derivates, polyphenols from any source, vitamin E and derivates, flavonoids & prenylated flavonoids, red salvia and any cryptotanshinone containing extract, bisabolol, N-acetylneuraminic acid, any DNA repair enzyme, creatine, Sechium edule extract, sericoside and derivates, green tea extract and more generally all tea species, niacinamide and derivates, any resorcinol and derivate including phenylethyl resorcinol, glutathione and derivates, Nymphaea Caerulea Flower Extract (and)/or Nelumbo Nucifera Flower Extract and more generally any lotus and/or waterlily extract, baicalin, baicalein, tryptophane, quercetin and derivates, any jasmine extract, any microorganism extract and/or ferment and/or supernatant, any antioxidant enzyme like SOD or catalase and others, any anti cell senescence active ingredient, baicaline, baicaleine, azelaic acid and derivates, cysteamine and derivates, carnosine and derivates such as carcinine, all microcirculation boosters like alpha glucosyl hesperidine and others, tangerine peel extract, any peptide of any source and in particular palmitoyl tripeptide-5 and/or acetyl dipeptide-1 cetyl ester, any AHA, Salicylic acid and derivates, adenosine and derivates, astaxanthin and derivates, hyaluronic acid and derivates, saccharide isomerate, ceramides and derivates and precursors, any oil, Ectoin, Ginkgo biloba leaf extract and other extracts, scutellaria extract, any Epilobium extract, any Rhododendron extract, Ectoin and derivates, elderberry flower extract, Kigelia africana extract of any part of the plant including fruit, any soy extract, any sweet pea extract, any vegetal peptide.

15. A cationic micro-vesicle according to claim 14, wherein the active agent is melatonin.
